(19) United States
(12) Patent Application Publication
(10) Pub. No.: US 2023/0326545 A1
(43) Pub. Date: Oct. 12, 2023

(54) SYSTEM AND METHOD FOR PREDICTING BIOLOGICAL ACTIVITY OF CHEMICAL OR BIOLOGICAL MOLECULES AND EVIDENCE THEREOF

(71) Applicant: PEPTRIS TECHNOLOGIES PRIVATE LIMITED, Bangalore (IN)

(72) Inventors: Venkatasubramanian Narayanan, Bangalore (IN); Anand Budni, Bangalore (IN); Amit Mahajan, Bangalore (IN)

(21) Appl. No.: 18/026,809

(22) PCT Filed: Sep. 14, 2021
(86) PCT No.: PCT/IN2021/050903

§ 371 Date: Mar. 16, 2023

(30) Foreign Application Priority Data
Sep. 18, 2020 (IN) 202041040578

(51) Int. Cl. G16B 15/30; G16B 40/20

(52) U.S. Cl. CPC G16B 15/30; G16B 40/20

(57) ABSTRACT

A system 100 for predicting binding affinity of chemical or biological molecules and their protein targets and generating pair-wise attention map as an evidence of binding between the chemical or biological molecules and their protein targets is provided. The system 100 includes a binding activity predicting system 104 receives the knowledge data of the chemical or biological molecules and their protein targets from the global knowledge database 102 and processes the knowledge data to convert into tokens of proteins and tokens of molecules. The tokens of protein and tokens of molecules are used to train a protein and molecule representation model to predict biological activity. The protein and molecule representation model is used to train a binding activity prediction model to predict binding affinities and to generate pair-wise attention maps as likelihoods of biological activity between amino acid residues and fragments involved in binding.

### BACKGROUND

### Technical Field

The embodiments herein generally relate to prediction of biological activity of molecules, and more particularly to a system and method for predicting binding affinity between chemical or biological molecules and their protein targets and generating an evidence of biological activity using machine learning models.

### Description of the Related Art

Determination of protein-protein interaction and protein-small molecule interaction, especially, in the area of drug discovery, is a challenging and cumbersome process as there are many possible degrees and ways of binding of proteins with a large number of possible molecules. One of the biggest challenges in trying to predict binding affinities is the complexity of interactions, for example, which regions of molecules involved in binding between the interacting chemical or biological molecules and their protein targets. Further, experimentally observed data about binding affinity between the chemical or biological molecules and their protein targets is sparsely populated. Hence, the experimentally observed data about binding affinity is not accessible to everyone for further analysis and research. Also, experimental observations would require a lot of effort and time, and with the huge available space of possible molecules, it is nearly impossible to device experimental methods to ascertain the binding affinities.

Various binding affinity prediction tools have been widely available in the market for some time. These tools rely on manual curation of protein and chemical molecule data such as three dimensional (3D) structure approximation and SMILES strings. Some conventional approaches rely on three dimension (3D) structural information of protein. Once the 3D structural information of protein is obtained, the small molecules are processed and docked with the protein to fit the shape or to some of the regions of the protein to predict the binding affinities using a minimized energy model. However, the conventional approaches and/or predicted structural data may not be adequate for working with novel proteins and would fail in matching binding affinity accurately. Moreover, it is hard to predict the 3D structure of the protein from the protein sequence and there may be some regions of protein in disordered state, as the protein may change its shape easily.

In some conventional approaches, ligand based, and structure based virtual screening are used to shortlist compounds. These methods are time consuming and lack the generalization and accuracy. Hence, an effective way of predicting binding affinities is still needed.

Accordingly, there remains a need to address the aforementioned technical drawbacks in existing technologies in predicting binding affinity between molecules.

### SUMMARY

In view of the foregoing, an embodiment herein provides a method for predicting binding affinity between at least one of a chemical or a biological molecule and its protein target using a binding activity predicting system. The method includes (i) pre-processing the knowledge data of a chemical or a biological molecule and its protein targets, (ii) converting the protein data into tokens of proteins, (iii) converting the molecule data into tokens of molecules by grouping substructures of the molecule using unique tokens, (iv) providing the tokens of molecules and the tokens of proteins to train a first machine learning model for generating a protein and molecule representation model in order to learn protein and molecule representations, (v) processing the binding activity data for a pair of a known protein and a known molecule to convert into tokens of the known protein and tokens of known molecule respectively, (vi) generating, using the protein and molecule representation model, embeddings for the known protein and the known molecule in the tokens of known protein and the tokens of known molecules, (vii) training a second machine learning model to generate a binding activity prediction model to predict a binding affinity and to generate pairwise attention maps between amino acid residues and atoms involved in binding, (viii) predicting, using at least one of the protein and molecule representation model or the binding activity prediction model, the binding affinity of amino acid residues of a test protein and fragments of a test molecule when the test protein and test molecule is provided as an input to the at least one of the protein and molecule representation model or the binding activity prediction model, (ix) generating, using at least one of the protein and molecule representation model or the binding activity prediction model, a pairwise attention map representing the amino acid residues of the test protein and the fragment of the test molecule involved in binding. The pre-processing of the knowledge data of the chemical or the biological molecule and its protein targets includes at least one of (i) correcting outliers, (ii) identifying missing data, (iii) determining latent relationships between different attributes of dataset to obtain a protein data, a molecule data and a binding activity data or (iv) data augmentation.

In some embodiments, the method includes (i) receiving the knowledge data of the chemical or the biological molecule and its protein target from a device including a global knowledge database, and (ii) storing the knowledge data of the chemical or biological molecule and its protein target in a database of a binding activity predicting system. The binding activity predicting system are communicatively connected to the device.

In some embodiments, the protein data includes pre-processing data including at least one of protein sequences, annotated proteins or un-annotated proteins. The molecule data includes pre-processed data of at least one of chemical compounds, biochemical compounds, chemical structures, crystal structures of chemicals or chemical reaction.

In some embodiments, the protein data is converted into the tokens of proteins by (i) annotating amino acid sequences of the protein at conserved or catalytic or binding site, (ii) predicting a secondary structure of the amino acid sequences, (iii) predicting a solvent accessibility of the amino acid sequences, and (iv) converting the amino acid sequences of the protein into the tokens of the protein.

In some embodiments, the substructures of the molecule are grouped, using at least one of a fragment type and properties prediction tool or a graph structure encoding tool, by (i) creating a set of substructures based on molecule data analysis (ii) creating one or more fragments by cleaving the molecule at the bonds of the molecule, and (iii) converting loop identifiers into the unique tokens.

In some embodiments, the global knowledge database includes a universal protein resource (UNIPROT), a protein data bank (PDB), ZINC, ChEMBL and Binding Database (BINDINGDB).

In some embodiments, the molecules data includes data in a Simplified Molecular Input Line Entry System (SMILES) format.

In some embodiments, the tokens of protein includes information of an amino acid type, amino acid annotations and properties of protein. The token of molecule includes information of properties of fragments in the molecule and fragment types.

In some embodiments, the binding activity data includes pre-processed data of at least one of experimental observed binding data, binding assay data and observed protein-ligand complexes. The binding activity data includes data of the already proven binding affinity between proteins and molecules.

In some embodiments, the pair-wise attention maps includes an evidence for at least one of (a) an amino acid fragment or sub-sequences of the protein which is taking part in the binding activity, (b) a set of binding residues from the protein sequence, c) a fragment of the molecule that is taking part in the activity, (d) a map of the molecule fragment to sub-sequences of the protein taking part on the activity, or (e) a map of fragments of the molecules to residues in the protein sequence.

In some embodiments, the method includes implementing at least one of (i) one or more of traditional deterministic reasoning techniques, (ii) data-modelling using ontologies and knowledge inference rules, and (iii) machine learning techniques, for pre-processing the protein data and the molecule data.

In some embodiments, the second machine learning model is trained using the protein and molecule representation model to generate the binding activity prediction model. The binding activity prediction model includes a deep learning model or a neural network model. The binding activity prediction model is trained using a supervised method.

In some embodiments, the protein and molecule representation model includes a deep learning model or a neural network model. The protein and molecule representation model is trained using an unsupervised method. The unsupervised method includes a masked language model or an autoregressive model.

In an aspect, an embodiment herein provides a system for predicting binding affinity between at least one of a chemical or a biological molecule and its protein target using a binding activity predicting system. The system includes a processor that (i) pre-processes the knowledge data of a chemical or a biological molecule and its protein targets, (ii) converts the protein data into tokens of proteins, (iii) converts the molecule data into tokens of molecules by grouping substructures of the molecule using unique tokens, (iv) provides the tokens of molecules and the tokens of proteins to train a first machine learning model for generating a protein and molecule representation model in order to learn protein and molecule representations, (v) processes the binding activity data for a pair of a known protein and a known molecule to convert into tokens of the known protein and tokens of known molecule respectively, (vi) generates, using the protein and molecule representation model, embeddings for the known protein and the known molecule in the tokens of known protein and the tokens of known molecules, (vii) trains a second machine learning model to generate a binding activity prediction model to predict a binding affinity and to generate pairwise attention maps between amino acid residues and atoms involved in binding, (viii) predicts, using at least one of the protein and molecule representation model or the binding activity prediction model, the binding affinity of amino acid residues of a test protein and fragments of a test molecule when the test protein and test molecule is provided as an input to the at least one of the protein and molecule representation model or the binding activity prediction model, (ix) generates, using at least one of the protein and molecule representation model or the binding activity prediction model, a pairwise attention map representing the amino acid residues of the test protein and the fragment of the test molecule involved in binding. The pre-process of the knowledge data of the chemical or the biological molecule and its protein targets includes at least one of (i) correcting outliers, (ii) identifying missing data, (iii) determining latent relationships between different attributes of dataset to obtain a protein data, a molecule data and a binding activity data or (iv) data augmentation.

The binding activity predicting system predicts variety of properties and activity for proteins. The predictions of the binding activity predicting system are far superior and more accurate. The binding activity predicting system screens against millions of compounds for activity and specificity.

### BRIEF DESCRIPTION OF THE DRAWINGS

The embodiments herein will be better understood from the following detailed description with reference to the drawings, in which:

FIG. 1 illustrates a system for predicting a biological activity of chemical or biological molecules and their protein targets and generating a pair-wise attention map as an evidence of the biological activity between the chemical or biological molecules and their protein targets according to an embodiment herein;

FIG. 2 is an exploded view of a binding activity predicting system of FIG. 1 according to an embodiment herein;

FIGS. 3A and 3B are flow diagrams that illustrate a method of predicting binding affinity of chemical or biological molecules and their protein targets and generating a pair-wise attention map as an evidence of binding between the chemical or biological molecules and their protein targets using a binding activity predicting system of FIG. 1 according to an embodiment herein;

FIG. 4 is an exemplary graphical representation that represents a linear map of activity of parts of chemical or biological molecules and their protein targets according to an embodiment herein;

FIG. 5A illustrates an exemplary semantic representation of a target activity generated using the binding activity predicting system of FIG. 1 according to an embodiment herein;

FIG. 5B is an exemplary Database of Useful Decoys-Enhanced (DUDE) results of machine learning/Artificial intelligence (AI) platform that is implemented in the binding activity predicting system of FIG. 1 according to an embodiment herein;

FIG. 6 is an exemplary distribution of predicted activity for 30 targets from a DUDE dataset according to an embodiment herein; and

FIG. 7 is a schematic diagram of a computer architecture of binding affinity predicting system that is configured to perform any one or more of the methodologies herein in accordance with the embodiments herein.

### DETAILED DESCRIPTION OF THE DRAWINGS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

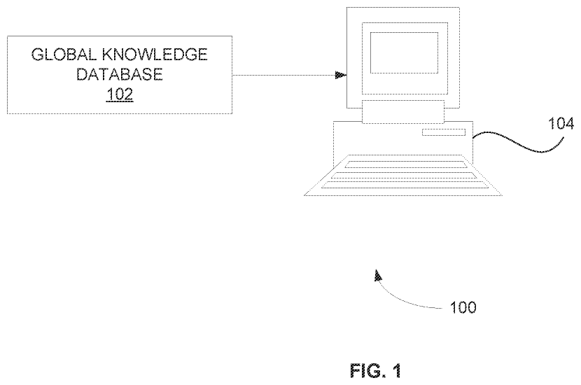

As mentioned, there remains a need for a system and method for predicting binding affinity between chemical or biological molecules and their protein targets in a fast and accurate manner without relying on experimentally verified information about 3D structure of proteins. Various embodiments disclosed herein provide a system and method for predicting binding affinity of chemical or biological molecules and their protein targets and generating a pair-wise attention map as an evidence of binding between the chemical or biological molecules and their protein targets using a machine learning model. Referring now to the drawings, and more particularly to FIGS. 1 through 7, where similar reference characters denote corresponding features consistently throughout the figures, preferred embodiments are shown.

FIG. 1 illustrates a system for predicting a biological activity of chemical or biological molecules and their protein targets and generating a pair-wise attention map as an evidence of the biological activity between the chemical or biological molecules and their protein targets according to an embodiment herein. The binding affinity of chemical or biological molecules and their protein targets may be a binding affinity between a protein and a molecule. Proteins are large biomolecules, or macromolecules, consisting of one or more long chains of amino acid residues. The molecule may include peptides, proteins and chemically synthesised molecules. The molecules may be a biological compound, a low molecular weight organic compound, a small molecule chemical compound or natural compounds. The molecule may be a biological compound, a small molecule, a low molecular weight organic compound, a chemical compound or a drug. The system 100 includes a global knowledge database 102 and a binding activity predicting system 104. The binding activity predicting system 104 includes a memory and a processor. The memory stores a database. A user may collect large number of knowledge data of the chemical or biological molecules and their protein targets from the global knowledge database 102 and provide the knowledge data of the chemical or biological molecules and their protein targets to the binding activity predicting system 104 for training machine learning models to predict protein and molecule representations, which in turn used in predicting binding affinities between chemical or biological molecules and their protein targets and in generating a pair-wise attention map of the chemical or biological molecules and their protein targets. In some embodiment, the binding activity predicting system 104 automatically receives the knowledge data of the chemical or biological molecules and their protein targets from the global knowledge database 102 through a network. The network may be a wireless network, a wired network, a combination of a wireless network and wired network or an Internet.

The global knowledge database 102 may include universal protein resource (UNIPROT), protein data bank (PDB), ZINC, ChEMBL and Binding Database (BINDINGDB). The knowledge data of the chemical or biological molecules and their protein targets may include protein sequence data, annotated data of proteins, un-annotated data of proteins, molecules information (includes chemical data), binding assay data, experimental observed binding data and observed protein-ligand complexes. The binding activity predicting system 104 may be a handheld device, a mobile phone, a PDA (Personal Digital Assistant), a tablet, a computer, an electronic notebook or a smartphone.

The binding activity predicting system 104 receives the knowledge data of the chemical or biological molecules and their protein targets from the global knowledge database 102 and stores the knowledge data of the chemical or biological molecules and their protein targets in the database of the binding activity predicting system 104. The binding activity predicting system 104 creates a training dataset from the knowledge data of the chemical or biological molecules and their protein targets by processing the knowledge data of the chemical or biological molecules and their protein targets stored in the database of the binding activity predicting system 104.

The binding activity predicting system 104 pre-processes the knowledge data of the chemical or biological molecules and their protein targets for (i) correcting outliers, (ii) dealing with missing data and, (iii) discovering latent relationships between different attributes of dataset and obtains protein data, molecules data and binding activity data. The protein data may include pre-processed data of at least one protein sequences, annotated proteins and un-annotated proteins. The molecules data may include pre-processed data of at least one of chemical compounds, biochemical compounds, chemical structures, crystal structures of chemicals and chemical reaction. The molecules data may be in Simplified Molecular Input Line Entry System (SMILES) format. The binding activity predicting system 104 further pre-processes the protein data and the molecules data to convert (i) the protein data into tokens of protein, and (ii) the molecules data into tokens of molecules.

The tokens of protein may include information of amino acid residues as words. The tokens of protein may include information of amino acid type, amino acid annotations and properties of proteins as words. The properties of the proteins may include a secondary structure, binding sites, a shape, and a solvent accessibility. The binding activity predicting system 104 may receive input of the amino acid type of the proteins. The binding activity predicting system 104 may use INTERPRO for amino acid annotation of the proteins. The binding activity predicting system 104 may predict the secondary structure of the proteins using protein structure prediction tools known in the art. The binding activity predicting system 104 may use a Hydrogen bond estimation algorithm (e.g. DSSP) to predict the secondary structure. The binding activity predicting system 104 may use neural networks to predict the secondary structures and solvent accessibility of the proteins. The neural networks may be a built-in predictor or predictors known in the art. In some embodiments, the protein data is converted into the tokens of proteins by (i) annotating amino acid sequences of the protein at conserved or catalytic or binding site, (ii) predicting a secondary structure of the amino acid sequences, (iii) predicting a solvent accessibility of the amino acid sequences, and (iv) converting the amino acid sequences of the protein into the tokens of the protein.

The tokens of molecules may include information of fragments in molecules as words. The tokens of molecules may include information of properties of fragments in the molecules and fragment types and properties thereof. The molecules data may be converted into the tokens of molecules using fragment types and properties prediction tools and graph structure encoding tools that encode the molecules as a sequence of fragments tokens including the properties thereof. The properties of fragments in the molecules may include a structure, a molecular weight, and a solubility.

The binding activity predicting system 104 may use one or more of traditional deterministic reasoning techniques, data-modelling using ontologies and knowledge inference rules and machine learning techniques (such as classification and clustering) to pre-process the protein data and the molecules data.

The binding activity predicting system 104 uses the tokens of protein and the tokens of molecules in molecules as the training dataset to train a first machine learning model to learn protein and molecule representations for obtaining a protein and molecule representation model. The protein and molecule representations may represent matching of known properties of the proteins and the molecules. The protein and molecule representation model may be a deep learning model or a neural network model. The protein and molecule representation model may be trained using unsupervised methods. The unsupervised methods may include a masked language model and an autoregressive model.

The binding activity predicting system 104 pre-processes the binding activity data for a known pair of a protein and molecule to convert into tokens of protein and tokens of molecules respectively. The binding activity data may include pre-processed data of at least one experimental observed binding data, binding assay data and observed protein-ligand complexes. The binding activity predicting system 104 uses the protein and molecule representation model to generate embeddings for the protein and the molecule separately or combinedly.

In some embodiments, the binding activity predicting system 104 uses the protein and molecule representation model to train a second machine learning model to obtain a binding activity prediction model. The binding activity prediction model predicts binding affinities between the amino acid residues and the fragments and generates pair-wise attention maps between the amino acid residues and the fragments involved in binding. The binding activity prediction model may be a deep learning model or a neural network model. The binding activity prediction model may be trained using supervised methods.

The binding activity predicting system 104 predicts the binding affinity and generates the pair wise attention map for test data using the binding activity prediction model, when the test data is provided as input to the binding activity prediction model. The test data may be at least one of unknown protein, unknown molecule or any other related data. The pair-wise attention map represents which fragments of molecules and amino acid residues are involved, and their properties, in binding and/or training. In some embodiments, the test protein and test molecule is provided as an input to the at least one of the protein and molecule representation model or the binding activity prediction model. The pair-wise attention map may provide the evidence for a) a segment/subsequence of the protein or amino acids which is taking part in the binding activity; b) a set of binding amino acid residues from the protein sequence; c) a fragment of the molecule that is taking part in the activity; d) a map of the molecule fragment to subsequences of the protein taking part on the activity and e) a map of fragments in the molecules to amino acid residues in the protein sequence.

In some embodiments, the binding activity predicting system 104 performs ADME (Absorption, Distribution, Metabolism and Excretion) prediction which is a series of predictions for activity with protein targets that are critical in Absorption, Distribution, Metabolism and Excretion processes within the human body. This ADME prediction ensures that a drug has a right bioavailability and has an improved efficacy. In some embodiments, the binding activity predicting system 104 performs off-target effects using the machine learning models where the binding activity predicting system 104 screens against a panel of targets other than the main target of interest for the drug, thereby ensuring that possible side-effects and adverse reaction can be predicted early for the drug more accurately. In some embodiments, the binding activity predicting system 104 predicts the molecule properties including solubility, lipophilicity, etc.

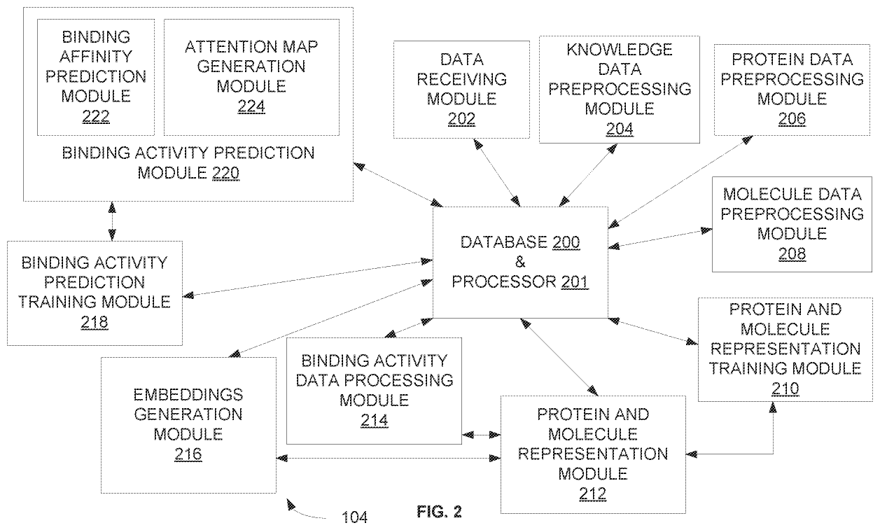

FIG. 2 is an exploded view of a binding activity predicting system of FIG. 1 according to an embodiment herein. The binding activity predicting system 104 includes a memory that stores a database 200, a processor 201, a data receiving module 202, a knowledge data pre-processing module 204, a protein data pre-processing module 206, a molecule data pre-processing module 208, a protein and molecule representation training module 210, a protein and molecule representation model 212, a binding activity data processing module 214, an embeddings generation module 216, a binding activity prediction training module 218 and a binding activity prediction model 220. The binding activity prediction model 220 includes a binding affinity prediction module 222 and an attention map generation module 224.

The data receiving module 202 receives knowledge data of chemical or biological molecules and their protein targets from the global knowledge database 102 and stores the knowledge data of the chemical or biological molecules and their protein targets in the database 200. The global knowledge database 102 may include universal protein resource (UNIPROT), protein data bank (PDB), ZINC, ChEMBL and Binding Database (BINDINGDB). The knowledge data of the chemical or biological molecules and their protein targets may include protein sequence data, annotated data of proteins, un-annotated data of proteins, molecules data (includes chemical data), binding assay data, experimental observed binding data and observed protein-ligand complexes. The chemical or biological molecules and their protein targets may include proteins and molecules. The molecules may be biological compounds, small molecules, low molecular weight organic compounds, chemical compounds or drugs. The data receiving module 202 may receive the knowledge data of the chemical or biological molecules and their protein targets from the global knowledge database 102 either through a user or through a network automatically. The network may be a wireless network, a wired network, a combination of a wireless network and a wired network or an Internet.

The knowledge data pre-processing module 204 pre-processes the knowledge data of the chemical or biological molecules and their protein targets for (i) correcting outliers, (ii) dealing with missing data and (iii) discovering latent relationships between different attributes of dataset and obtains protein data, molecules data and binding activity data. The protein data may include pre-processed data of at least one protein sequences, annotated proteins and un-annotated proteins. The molecules data may include pre-processed data of at least one chemical compounds, biochemical compounds, chemical structures, crystal structures of chemicals and chemical reaction. The molecules data may be in Simplified Molecular Input Line Entry System (SMILES) format. The binding activity data may include pre-processed data of at least one experimental observed binding data, binding assay data and observed protein-ligand complexes.

The protein data pre-processing module 206 pre-processes the protein data and converts the protein data into tokens of protein. The tokens of protein may include information of amino acid residues as words. The tokens of protein may include information of amino acid type, amino acid annotations and properties of proteins as words. The properties of the proteins may include a secondary structure, binding sites, a shape, and a solvent accessibility. The protein data pre-processing module 206 may use an input of the amino acid types included in the protein. The protein data pre-processing module 206 may process the amino acid annotation of the proteins using amino acid annotation tools known in the art. The binding activity predicting system 104 may use INTERPRO for amino acid annotation of the proteins. The protein data pre-processing module 206 may use a Hydrogen bond estimation algorithm (e.g. DSSP) to predict the secondary structure. The protein data pre-processing module 206 may use neural networks to predict the secondary structures and solvent accessibility of the proteins. The protein data pre-processing module 206 may use one or more of traditional deterministic reasoning techniques, data-modelling using ontologies and knowledge inference rules and machine learning techniques etc. (such as classification and clustering) to pre-process the protein sequence data.

In some exemplary embodiments, a protein is converted into tokens of protein (words) using various protein data processing tools. For example, the amino acid sequence of the protein may be represented as <MACDESPPETWY> using Planton, in which each letter indicates type of amino acid among the total 20 amino acids. The predicted amino acid sequence may be annotated with conserved sites or catalytic sites or binding site using INTERPRO or such methods. The secondary structure of the amino acid sequence may be predicted using a Hydrogen bond estimation algorithm (e.g. DSSP). The solvent accessibility of the amino acid sequence may be predicted using neural networks. The secondary structures may be predicted into three types such as Helix, beta sheet and coil. The solvent accessibility may be converted into two levels such as buried and exposed. The amino acid sequence, <MACDESPPETWY> may be converted into a tokens of proteins, <Helix>MCAD<Beta>ESPpeTWY. The tokens of proteins may start with the secondary structure, followed by the solvent accessibility of every amino acid residues. In the tokens of proteins, capital letter may indicate exposed and small letter may indicate buried. The tokens of proteins may also include information such as conserved sites, binding sites, etc.

The molecule data pre-processing module 208 pre-processes the molecules data and converts the molecules data into tokens of molecules. The tokens of molecules may include information of fragments in molecules as words. The tokens of molecules may include information of properties of fragments in the molecules and fragment types. The molecule data pre-processing module 208 may use fragment types and properties prediction tools and graph structure encoding tools to convert the molecules data into the tokens of molecules. The properties of fragments in the molecules may include a structure, a molecular weight, and a solubility.

The molecule data pre-processing module 208 may use one or more of traditional deterministic reasoning techniques, data-modelling using ontologies and knowledge inference rules and machine learning techniques (such as classification and clustering) to pre-process the molecules data.

In some exemplary embodiments, a molecule in SMILES syntax is converted into tokens of molecules using various molecule data processing tools. The molecule data processing tools may process the molecule by grouping substructures of the molecule using a unique token, for example, [*]—[O]—[CH3]). For grouping the substructures of the molecule, a set of substructures may be created based on large data analysis using ZINC database and one or more fragments may be created by cleaving the molecule at the bonds of the molecule. A branch in the molecule may be indicated using ‘(‘and’)’ as branch tokens. Loop connections in the molecule may be marked by converting the loop identifiers in the SMILES syntax into unique identifiers.

For example, Molecule, [CH3]-[C@H](—[NH2])-[CH2]-[N]1-[CH2]-[CH2]-[N](—[S](—[NH2])(32 [O])═[O])— [CH2]-[CH2]-1 with identified fragments such as FRAG1=[*]—[N](—[*])—[CH3] and FRAG2=[*]—[C](═[O])—[CH2]-[CH2]-[*] may be encoded as J-[FRAG1*]-D-[FRAG2*]-SJQQ, where, [NH2] may be encoded as J, [CH2] may be encoded as D and [═O] may be encoded as Q and FRAG1 may be [*]—[N](—[*])—[CH3] and FRAG2 may be [*]—[C](═[O])—[CH2]-[CH2]-[*].

The protein and molecule representation training module 210 matches the preprocessed molecules data using the tokens of molecules and the preprocessed protein data using the tokens of amino acids as training set to train a first machine learning model. This trained first machine learning model is a protein and molecule representation model 212 that could predict protein-molecule representations. The protein-molecule representations may represent matching of known properties of proteins and molecules. The protein and molecule representation model 212 may be a deep learning model or a neural network model. The protein and molecule representation training module 210 may use unsupervised methods to train the protein and molecule representation model 212. The unsupervised methods may include a masked language model or an autoregressive model.

The binding activity data processing module 214 processes the binding activity data for a known pair of a protein and molecule to convert into tokens of protein and tokens of molecules. The binding activity data may include pre-processed data of at least one of experimental observed binding data, binding assay data and observed protein-ligand complexes. The binding activity data may include data of the already proven binding affinity between proteins and molecules. The embeddings generation module 216 generates embeddings for the protein and molecule in the tokens of protein and tokens of molecules, separately or combinedly using the protein and molecule representation model 212. In some embodiments, after generating the embeddings the protein and molecule representation model 212 include tokens of protein, tokens of molecules and binding activity data tokens.

The binding activity prediction training module 218 uses the protein and molecule representation model 212 to train a second machine learning model. This trained second machine learning model is the binding activity prediction model 220, that could predict binding activity of the protein and molecule. The binding activity prediction model 220 predicts the binding affinity of the protein and molecules at the binding affinity prediction module 222 and generates a pair wise attention map at the attention map generation module 224, for test data, when the test data is provided as input to the binding activity prediction model 220. The test data may be at least one of unknown protein, unknown molecule or any other related data.

The binding affinity prediction module 222 predicts the binding affinity of amino acid residues in the protein and fragments in the molecules. The attention map generation module 224 generates the pairwise attention maps between amino acid residues and molecule fragment involved in binding. The pair-wise attention maps may provide an evidence for a) an amino acid fragment or subsequences of the protein which is taking part in the binding activity, b) a set of binding residues from the protein sequence, c) a fragment of the molecule that is taking part in the activity, d) a map of the molecule fragment to subsequences of the protein taking part on the activity and e) a map of fragments of the molecules to residues in the protein sequence.

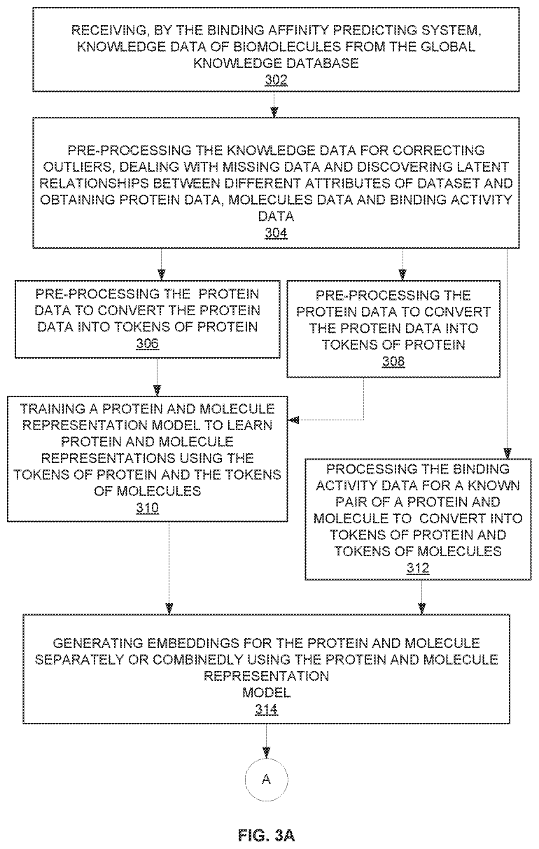

FIGS. 3A and 3B are flow diagrams that illustrate a method of predicting binding affinity of chemical or biological molecules and their protein targets and generating a pair-wise attention map as an evidence of binding between the chemical or biological molecules and their protein targets using a binding activity predicting system of FIG. 1 according to an embodiment herein. At step 302, large number of knowledge data of chemical or biological molecules and their protein targets is received from the global knowledge database 102 by the binding activity predicting system 104. The global knowledge database 102 may include universal protein resource (UNIPROT), protein data bank (PDB), ZINC, ChEMBL, and Binding Database (BINDINGDB). The knowledge data of the chemical or biological molecules and their protein targets may include protein sequence data, annotated data of proteins, un-annotated data of proteins, molecules data (includes chemical data), binding assay data experimental observed binding data, and observed protein-ligand complexes. The chemical or biological molecules and their protein targets may include proteins and molecules. The molecules may be biological compounds, small molecules, low molecular weight organic compounds, chemical compounds or a drugs. The binding activity predicting system 104 may receive the knowledge data of the chemical or biological molecules and their protein targets from the global knowledge database 102 either through a user or automatically through a network. The network may be a wireless network, a wired network, a combination of a wireless network and wired network or a Internet.

At step 304, the knowledge data of the chemical or biological molecules and their protein targets are pre-processed using the binding activity predicting system 104 for (i) correcting outliers, (ii) dealing with missing data and, (iii) discovering latent relationships between different attributes of dataset and protein data, molecules data and binding activity data are obtained. The protein data may include pre-processed data of at least one protein sequences, annotated proteins and un-annotated proteins. The molecules data may include pre-processed data of at least one chemical compounds, biochemical compounds, chemical structures, crystal structures of chemicals and chemical reaction. The molecules data may be in Simplified Molecular Input Line Entry System (SMILES) format.

At step 306, the protein data is further pre-processed by the binding activity predicting system 104 to convert the protein data into tokens of protein. The tokens of protein may include information of amino acid residues as words. The tokens of protein may include information of amino acid type, amino acid annotations and properties of proteins as words. The properties of the proteins may include a secondary structure, binding sites, a shape, and a solvent accessibility. The binding activity predicting system 104 may use one or more of traditional deterministic reasoning techniques, data-modelling using ontologies and knowledge inference rules and machine learning techniques (such as classification and clustering) to pre-process the protein data.

At step 308, the molecules data is pre-processed by the binding activity predicting system 104 to convert the molecules data into tokens of molecules. The tokens of molecules may include information of fragment in molecules as words. The tokens of molecules may include information of properties of fragments in the molecules and fragment types. The molecules data may be converted into the tokens of molecules using fragment types and properties prediction tools and graph structure encoding tools that encode the molecules as a sequence of atom tokens. The properties of fragments in the molecules may include a structure, a molecular weight, and a solubility. The binding activity predicting system 104 may use one or more of traditional deterministic reasoning techniques, data-modelling using ontologies and knowledge inference rules and machine learning techniques (such as classification and clustering) to pre-process the protein data and the molecules data.

At step 310, a protein and molecule representation model is trained to learn protein and molecule representations using the tokens of amino acids and the tokens of molecules in molecules as a training dataset. The protein and molecule representation model may be one or more of a neural network model or any other machine learning model. The protein and molecule representation model may be trained using unsupervised methods. The unsupervised methods may include a masked language model or an autoregressive model.

At step 312, the binding activity data is processed by the binding activity predicting system 104 for a known pair of a protein and molecule to convert into tokens of protein and tokens of molecules. The binding activity data may include pre-processed data of at least one experimental observed binding data, binding assay data and observed protein-ligand complexes. The experimental observed binding data may include data of the already proven binding affinity between proteins and the molecules. At step 314, embeddings for the protein and molecule are generated separately or combinedly using the protein and molecule representation model. After generating the embeddings, the protein and molecule representation model may include the tokens of protein, the tokens of molecules and binding activity data in tokens.

At step 316, a binding activity prediction model is trained using the protein and molecule representation model as a training dataset to predict binding affinities and to generate pairwise attention maps between amino acid residues of the proteins and fragments in molecules involved in binding. The binding activity prediction model may be one or more of a neural network model or any other machine learning model. The binding activity prediction model may be trained using supervised methods.

At step 318, the binding affinity is predicted, and the pair wise attention map is generated for test data using the binding activity prediction model, when the test data is provided as input to the binding activity prediction model. The test data may be at least one of unknown protein, unknown molecule or any other related data. The pair-wise attention maps may provide an evidence for a) a segment/subsequence of the protein or amino acids which is taking part in the binding activity; b) a set of binding residues from the protein sequence; c) fragments of the molecule that are taking part in the activity; d) a map of the molecule fragments to subsequences of the protein taking part on the activity and e) a map of fragments in the molecules to residues in the protein sequence.

The pair wise attention map may have weight or level of biological activity of different parts of the protein i.e. amino acid sequence as three dimensional representation. The sequence of protein may be represented at X axis and y-axis may represent different parts of molecules or molecule fragments. The pair wise attention map may be represented as a heat map with different level of biological activity shown in color coded manner. The heat map may be a three dimensional representation of the biological activity between the chemical or biological molecules and their protein targets.

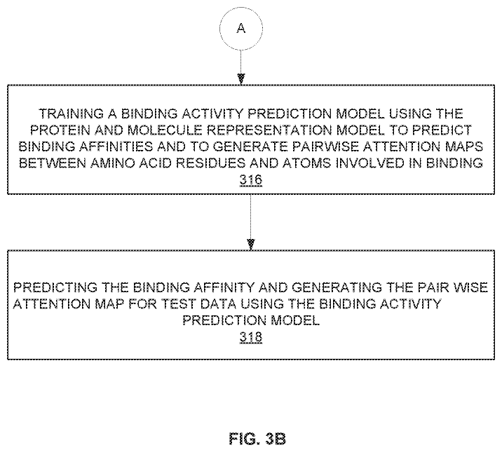

FIG. 4 is an exemplary graphical representation that represents a linear map of activity of parts of chemical or biological molecules and their protein targets according to an embodiment herein. The linear map represents the evidence of active fragments of the chemical or biological molecules or protein residues that are involved in biological activity. The Y-axis represents the relative importance of the residues as likelihoods (0.1 to 0.3 in the example) and the X axis represents the position of the amino acid in the primary sequence of the protein. In FIG. 4, 402 represents map of the protein residues that are involved in the activity and 404 represents linearly the activity of amino acids at different part of the protein molecules. The map shows evidence of the biological activity that helps verify the results achieved with the binding affinity prediction model. Different parts of the biological or chemical molecules or their protein targets may have different activity level.

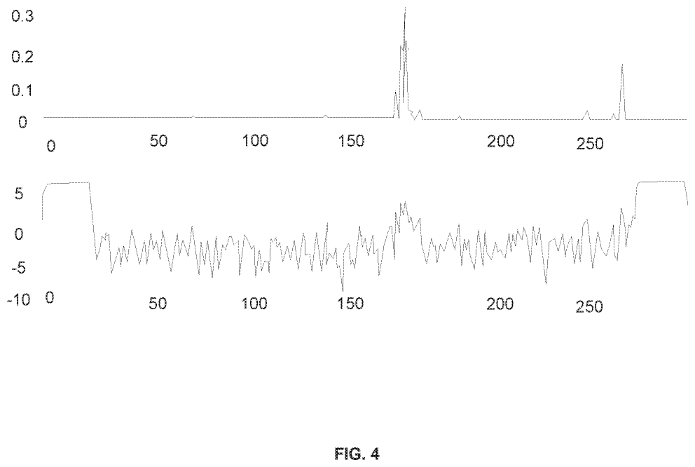

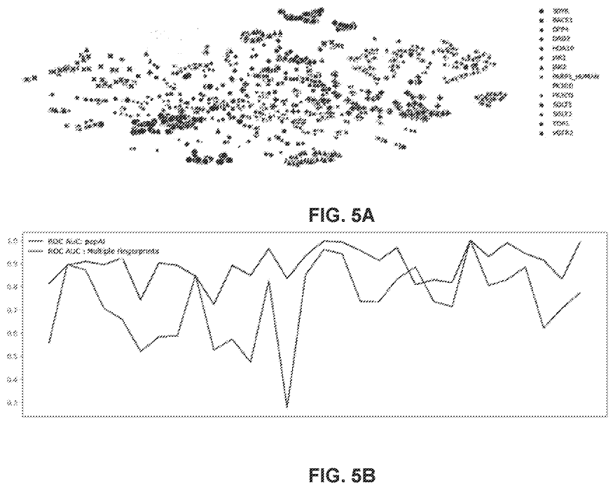

FIG. 5A illustrates an exemplary semantic representation of a target activity generated using the binding activity predicting system 104 of FIG. 1 according to an embodiment herein. The semantic representation may be a protein or molecule representation for a binding activity.

FIG. 5B is an exemplary Database of Useful Decoys-Enhanced (DUDE) results of machine learning/Artificial intelligence (AI) platform that is implemented in the binding activity predicting system 104 of FIG. 1 according to an embodiment herein. The binding activity predicting system 104 seamlessly fits within the existing discovery pipeline. DUDE results are a benchmark that requires the model to pick the active molecules from a large stack of similar decoy molecules.

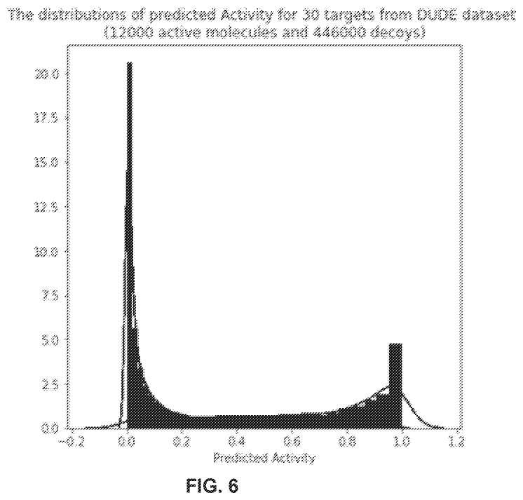

FIG. 6 is an exemplary distribution of predicted activity for 30 targets from a DUDE dataset according to an embodiment herein. DUDE is a well-known benchmark for structure-based virtual screening methods from the Shoichet Lab at UCSF. It is constructed by first gathering diverse sets of active molecules for a set of target proteins. A select set of exemplar actives is paired with a set of property matched decoys (PMD) and it serves as the test set for the model to differentiate between the true active and the decoy molecules. For a set of 12000 active pairs, the DUDE set contains 446000 decoy molecules that are property matched to the active set of molecules. In some embodiments, a significant number of (432000 out 446000, 96%) of the decoy molecules are predicted to have a very low activity according to the present binding activity predicting system 104. In some embodiments, greater than 9000 out of the 12000 active molecules are predicted by the binding activity predicting system 104.

The molecules are optionally represented as a simple SMILES string, a graph, a three dimensional (3D) object, a set of physio-chemical properties (fingerprints), or a bag of fragments and each of the representations are distilled using the machine learning model/architectures. A holistic semantic representation of the molecule predicts the activity with a protein that is derived. A protein representations includes an amino acid sequence, the evolutionary information, the functional classifications, domains, secondary structure and its allied properties. The binding activity predicting system 104 curates the protein and the molecule in a way to derive the best semantic representations. The machine learning models (e.g. a deep learning model) that are employed in the binding activity predicting system 104 are further custom created based on insights from approaches that have worked well in other domains. Optionally, the rigorously validated representations are used in further tasks like Activity prediction, ADMET and de-novo drug designs effectively.

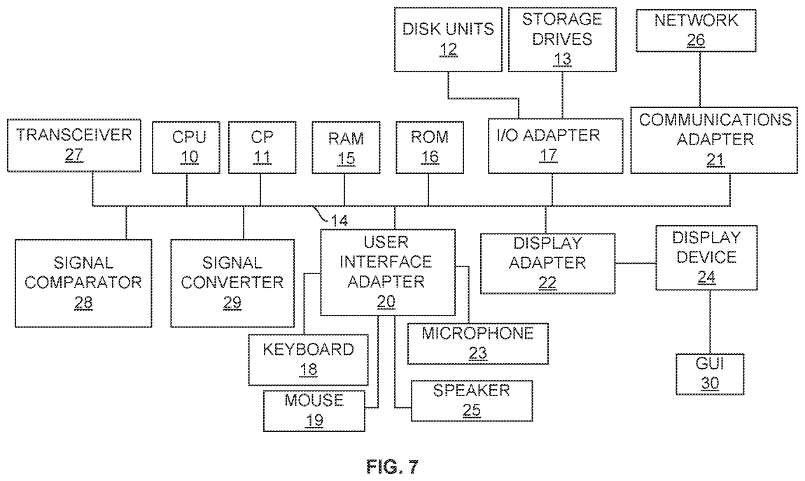

FIG. 7 is a schematic diagram of a computer architecture of binding affinity predicting system that is configured to perform any one or more of the methodologies herein in accordance with the embodiments herein. A representative hardware environment for practicing the embodiments herein is depicted in FIG. 5, with reference to FIGS. 1 through 4. This schematic drawing illustrates a hardware configuration of a server/computer system/computing device in accordance with the embodiments herein. The system includes at least one processing device CPU 10 that may be interconnected via system bus 14 to various devices such as a random access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The system 100 maps the protein sequence to activity without explicit use of 3D structure of the protein. The system 100 analyses a vast amount of data and applies transformation techniques (to convert into protein tokens and molecules tokens) on the data to enable and help machine learning algorithms to learn better. The system 100 performs semi-supervised and multi task methods to learn protein and molecule representations, hence accuracy is improved. For example, the system 100 uses the masked language model that may use context words surrounding a [MASK] token to try to predict what the [MASK] word should be, thereby improves the accuracy of the prediction. When predicting the binding affinity between proteins and molecules it is particularly important to know the region of protein involved in binding, this information could be used for various other methods to study target specificity, effectiveness or could also be used to verify with other industry methods to improve the confidence of predictions. The system 100 generates attention map that provides the biological activity of binding between chemical or biological molecules and proteins by providing likelihood information on the region of proteins and molecules involved in binding. The system 100 uses only protein sequence and molecule SMILES string/syntax as inputs and hence is applicable in wide variety of studies and applications. Since the proteins and molecules are transformed into tokens/words, the prediction model of the system 100 can be used to predict protein-protein interactions, protein-molecule interactions, etc.

The foregoing description of the specific embodiments will so fully reveal the general nature of the embodiments herein that others can, by applying current knowledge, readily modify and/or adapt for various applications without departing from the generic concept, and, therefore, such adaptations and modifications should be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method for predicting binding affinity between at least one of a chemical or a biological molecule and its protein target using a binding activity predicting system, wherein the method comprises,
pre-processing the knowledge data of a chemical or a biological molecule and its protein targets, wherein the pre-processing comprises at least one of (i) correcting outliers, (ii) identifying missing data, (iii) determining latent relationships between different attributes of dataset to obtain a protein data, a molecule data and a binding activity data or (iv) data augmentation;
converting the protein data into tokens of proteins;
converting the molecule data into tokens of molecules by grouping substructures of the molecule using unique tokens;
providing the tokens of molecules and the tokens of proteins to train a first machine learning model for generating a protein and molecule representation model in order to learn protein and molecule representations;
processing the binding activity data for a pair of a known protein and a known molecule to convert into tokens of the known protein and tokens of known molecule respectively;
generating, using the protein and molecule representation model, embeddings for the known protein and the known molecule in the tokens of known protein and the tokens of known molecules;
training a second machine learning model to generate a binding activity prediction model to predict a binding affinity and to generate pairwise attention maps between amino acid residues and atoms involved in binding;
predicting, using at least one of the protein and molecule representation model or the binding activity prediction model, the binding affinity of amino acid residues of a test protein and fragments of a test molecule when the test protein and test molecule is provided as an input to the at least one of the protein and molecule representation model or the binding activity prediction model; and
generating, using at least one of the protein and molecule representation model or the binding activity prediction model, a pairwise attention map representing the amino acid residues of the test protein and the fragment of the test molecule involved in binding.

2. The method as claimed in claim 1, wherein the method comprises
receiving the knowledge data of the chemical or the biological molecule and its protein target from a device comprising a global knowledge database, wherein the binding activity predicting system are communicatively connected to the device; and
storing the knowledge data of the chemical or biological molecule and its protein target in a database of a binding activity predicting system.

3. The method as claimed in claim 1, wherein the protein data comprises pre-processing data comprising at least one of protein sequences, annotated proteins or un-annotated proteins, wherein the molecule data comprises pre-processed data of at least one of chemical compounds, biochemical compounds, chemical structures, crystal structures of chemicals or chemical reaction.

4. The method as claimed in claim 1, wherein the protein data is converted into the tokens of proteins by (i) annotating amino acid sequences of the protein at conserved or catalytic or binding site, (ii) predicting a secondary structure of the amino acid sequences, (iii) predicting a solvent accessibility of the amino acid sequences, and (iv) converting the amino acid sequences of the protein into the tokens of the protein.

5. The method as claimed in claim 1, wherein the substructures of the molecule are grouped, using at least one of a fragment type and properties prediction tool or a graph structure encoding tool, by (i) creating a set of substructures based on molecule data analysis (ii) creating one or more fragments by cleaving the molecule at the bonds of the molecule, and (iii) converting loop identifiers into the unique tokens.

6. The method as claimed in claim 2, wherein the global knowledge database comprises a universal protein resource (UNIPROT), a protein data bank (PDB), ZINC, ChEMBL and Binding Database (BINDINGDB).

7. The method as claimed in claim 1, wherein the molecules data comprises data in a Simplified Molecular Input Line Entry System (SMILES) format.

8. The method as claimed in claim 1, wherein the tokens of protein comprise information of an amino acid type, amino acid annotations and properties of protein, wherein the tokens of molecule comprise information of properties of fragments in the molecule and fragment types.

9. The method as claimed in claim 1, wherein the binding activity data comprises pre-processed data of at least one of experimental observed binding data, binding assay data and observed protein-ligand complexes, wherein the binding activity data comprises data of the already proven binding affinity between proteins and molecules.

10. The method as claimed in claim 1, wherein the pair-wise attention maps comprises an evidence for at least one of (a) an amino acid fragment or sub-sequences of the protein which is taking part in the binding activity, (b) a set of binding residues from the protein sequence, c) a fragment of the molecule that is taking part in the activity, (d) a map of the molecule fragment to sub-sequences of the protein taking part on the activity, or (e) a map of fragments of the molecules to residues in the protein sequence.

11. The method as claimed in claim 1, wherein the method comprises implementing at least one of (i) one or more of traditional deterministic reasoning techniques, (ii) data-modelling using ontologies and knowledge inference rules, and (iii) machine learning techniques, for pre-processing the protein data and the molecule data.

12. The method as claimed in claim 1, wherein the second machine learning model is trained using the protein and molecule representation model to generate the binding activity prediction model, wherein the binding activity prediction model comprises a deep learning model or a neural network model, wherein the binding activity prediction model is trained using a supervised method.

13. The method as claimed in claim 1, wherein the protein and molecule representation model comprise a deep learning model or a neural network model, wherein the protein and molecule representation model is trained using an unsupervised method, wherein the unsupervised method comprises a masked language model or an autoregressive model.

14. A system for predicting binding affinity between at least one of a chemical or a biological molecule and its protein target using a binding activity predicting system, wherein the system comprises a processor that:
pre-processes the knowledge data of a chemical or a biological molecule and its protein targets, wherein the pre-processing comprises at least one of (i) correcting outliers, (ii) identifying missing data, (iii) determining latent relationships between different attributes of dataset to obtain a protein data, a molecule data and a binding activity data or (iv) data augmentation;
converts the protein data into tokens of proteins;
converts the molecule data into tokens of molecules by grouping substructures of the molecule using unique tokens;
provides the tokens of molecules and the tokens of proteins to train a first machine learning model for generating a protein and molecule representation model in order to learn protein and molecule representations;
processes the binding activity data for a pair of a known protein and a known molecule to convert into tokens of the known protein and tokens of known molecule respectively;
generates, using the protein and molecule representation model, embeddings for the known protein and the known molecule in the tokens of known protein and the tokens of known molecules;
trains a second machine learning model to generate a binding activity prediction model to predict a binding affinity and to generate pairwise attention maps between amino acid residues and atoms involved in binding;
predicts, using at least one of the protein and molecule representation model or the binding activity prediction model, the binding affinity of amino acid residues of a test protein and fragments of a test molecule when the test protein and test molecule is provided as an input to the at least one of the protein and molecule representation model or the binding activity prediction model; and
generates, using at least one of the protein and molecule representation model or the binding activity prediction model, a pairwise attention map representing the amino acid residues of the test protein and the fragment of the test molecule involved in binding.
